(19) United States
(12) Patent Application Publication
(10) Pub. No.: US 2025/0215369 A1
(43) Pub. Date: Jul. 3, 2025

(54) Emerse Bioreactor

(71) Applicant: Hochschule Kaiserslautern (Kaiserslautern University), Kaiserslautern (DE)

(72) Inventor: Michael Lakatos, Kaiserslautern (DE)

(73) Assignee: Hochschule Kaiserslautern (Kaiserslautern University), Kaiserslautern (DE)

(21) Appl. No.: 17/769,669

(22) PCT Filed: Oct. 14, 2020
(86) PCT No.: PCT/EP2020/078969

§ 371 Date: Aug. 30, 2024

(30) Foreign Application Priority Data
Oct. 15, 2019 (DE) 102019007167.0

(51) Int. Cl. C12M 1/00; C12M 1/12; C12M 1/26

(52) U.S. Cl. CPC C12M 21/02; C12M 23/04; C12M 23/22; C12M 25/18; C12M 29/06; C12M 31/08; C12M 33/00

(57) ABSTRACT

The invention relates to a bioreactor (1) comprising multiple substrates (2) made of solid or a semisolid material for the emerse growing of aerophilic, photosynthesizing organisms (3) for use with a light device (6) for providing light (60) for the organisms (3), wherein the bioreactor has a reactor chamber (10) inside which the substrates (2) for growing the organisms (3) are arranged, a gassing device (4) for supplying the organisms (3) with gas (40), and a humidifying device (5) for supplying the organisms (3) with moisture (50), wherein the substrates (2) are formed in pieces and are provided as bulk material (20) in the reaction chamber (10) during operation, wherein through-paths (21) for gas (40) and/or moisture (50) are provided between multiple substrates (2) and the bioreactor (1) is arranged in such a way that the bulk material (20) can be illuminated with light (60) from the light device (6). The invention also relates to a substrate for a bioreactor, a method for generating a biofilm and a use of the biofilm for fertilizing, for upgrading soil or for directly or indirectly feeding people or animals.

The present invention relates to a bioreactor for the growth of organisms on substrates. The bioreactor is configured for use with a light device (6) for providing light to the organisms, The bioreactor comprises several substrates of a solid or semisolid material for emerse growth of aerophilic, photosynthesizing organisms. It further comprises a reactor chamber in the interior of which the substrates for growing the organisms are arranged, a gassing device for supplying the organisms with gas, a humidifying device for supplying the organisms with moisture, and a light device for providing light for the organisms.

Previously common methods for the production of photosynthesizing organisms and cells, such as cyanobacteria, algae, mosses, ferns and cell cultures, were carried out submersed, i.e., embedded or swirled in a nutrient medium.

On 8 Oct. 2016, Dr Björn Podola published the article “Ein neuer Ansatz zur wirtschaftlichen Nutzung von Mikroalgen” on the internet platform www.git-labor.de, in which it is proposed to insert microporous plates into a reactor chamber filled with water to make production more effective.

The submerse methods used so far essentially have the disadvantages of limited diffusion of carbon dioxide in the medium, clumping of the cultures, a growth-reducing ratio of oxygen to carbon dioxide, a constant energy input for doubling the medium and separating the cultivated material from the medium. Furthermore, only organisms that are adapted to live in water can be cultivated by submerse methods. In an emerse bioreactor, on the other hand, the biological reaction does not take place submerse, but gas-exposed (emerse) on a solid.

The publication of Dorina Strieth, Julia Schwing, Stephan Kuhne, Michael Lakatos, Kai Muffler, Roland Ulber: “A semi-continuous process based on an ePBR for the production of EPS using Trichocoleus sociatus” in Journal of Biotechnology, 256 (2017), 6-12, discloses an emerse bioreactor designed for the growth of terrestrial microorganisms on the inside of its reactor chamber. However, the disadvantage of this arrangement is that it only achieves a low productivity.

The object of the invention is to make the production of terrestrial aerophilic organisms in a reactor chamber more effective.

The subject matter of the invention is defined by the features of the independent claims. The subclaims relate to advantageous embodiments of the invention.

Beyond the prior art, the emerse bioreactor has substrates that are formed as pieces and are provided as bulk material in the reactor chamber during operation. Through-paths for gas and/or moisture are present between multiple substrates.

An advantage of such a bioreactor setup is that one or more biofilms can be generated from aerophilic growth with high volume efficiency and high growth rate. This results from the fact that the bulk material enables a large increase in the surface that can be used for the build-up of biofilms, particularly compared to the inner surface including the installations of the emerse reactor chamber by Strieth et al. according to prior art. In addition, the through-paths for gas and moisture in the bulk material in the emerse bioreactor allow the biofilm to be well supplied. A growth-promoting ratio of oxygen to carbon dioxide and a high diffusion of carbon dioxide in the reactor is preferred.

In this patent application, the size of the surface of the substrates is defined as an area which can be determined at least by approximate determination, the surface being determined from averaged external dimensions of the substrates or averaged internal dimensions of through-paths through the substrate(s). Porosity within the substrate(s) and surface roughness of the substrates are not considered.

An excessive enlargement of the usable surface area leads to increasingly smaller through-paths in a specified external volume so that the supply of the biofilm becomes suboptimal or even impossible if the enlargement is too high. A differential pressure that is applied along the through-paths also plays a role. In practice, however, it is advantageous to manage with low differential pressures. For example, a differential pressure of less than 104 kPa, preferably less than 10 kPa and particularly preferably less than 1 kPa is applied. One advantage of these pressure ratios is that they can also be provided by generating negative pressure when operating under the earth's atmospheric pressure, which is a maximum of 104 kPa. The ratio of substrate surface to container surface is preferably optimized for this. It is preferably at least ten, particularly preferably at least one to fifty. The container surface mentioned refers to the inner surface of the reactor chamber in which the substrate fill is placed.

For example, terrestrial eukaryotic algae, e.g., Chlamydomonas, Trentepohlia, and/or prokaryotic cyanobacteria e.g., Nostoc flagelliforme and/or lichens and/or fungi and/or mosses and/or ferns can be used as organisms. Cell cultures from higher and lower plants and/or plant cells can be used. Moreover, co-cultures from prokaryotes, e.g., bacteria and/or archaea, or fungi can be used together with the phototrophs. These can interact mutualistically and make better use together of the resources available in the bioreactor. In principle, the bioreactor is suitable for all aerophilic photosynthesizing organisms. Preferably, the bioreactor is operated in a sterile or approximately sterile or low-contamination manner.

The bioreactor can be used to produce substances such as polysaccharides, active substances, e.g. cyanovirin, nostoflan, noscomin, scytovirin, spirulan, scytonemin, dyes such as phycocyanin and/or phycoerythrin and/or allophycocyanin and/or chlorophyll and/or carotenoids as well as various lipids and proteins. These can be extracted from the biomass by down-streaming. Alternatively, the biomass can be directly utilized, for example as food or fertilizer or as an energy carrier.

In particular, the substrates have an average size of more than 1 mm, preferably more than 2 mm and more preferably more than 5 mm and preferably at most 50 mm and more preferably at most 20 mm.

With a substrate size of less than 1 mm, considerable pressure is needed to convey gas and moisture through the bulk material. For large reactors with reactor chambers with a dimension of more than 1 m, larger substrates can be used with a particular advantage, particularly substrates that are larger than 50 mm on average. In this case, the length of the through-paths is increased, which is achievable through an increased cross-section of the through-paths, which in turn is a consequence of larger substrates. Due to a large weight as a result of a large bulk material height, the through-paths can also be compressed in the case of less solid substrates. An average substrate size of more than 50 mm may be provided to prevent complete closure and to keep a sufficient through-cross-section open. Preferably, the size distribution of the substrates within the reactor chamber is at least approximately homogeneous.

Preferably, the substrates can absorb, store and release liquid. This can be achieved, for example, by a material of the substrate that can store the liquid in the material itself, such as hydrocolloids or (natural) fibers or fiber composite materials, or by cavities in the substrates, such as pores. Simultaneously with fluid-particularly water-, nutrients can also be stored. The substrates can alternatively or additionally include bio-stimulants, particularly in the liquid.

In this patent application, a semisolid is also understood to be a material that has a lower strength than usual solids but shows dimensional stability over a longer period. For example, a semisolid may be a gel that can absorb water, in particular a hydrogel. Types of gels such as organogels, particularly polymer gels, nanocomposite gels or aerogels can be applied. Preferably, the semisolid is dimensionally stable despite strong deformability and suffers only insignificant permanent deformations. The substrates may consist of the hydrogel at least to a large extent.

Solid substrates may include inorganic, particularly mineral, and/or organic material. The substrates may include nutrients for the organisms that can be dissolved from the substrates by liquid or the organisms themselves. Solid substrates in particular can have pores open towards the surface. Solid substrates can include, for example, glass, particularly porous glass, ceramics or plastic. For example, volcanic granules and/or clay-based material or absorber materials such as acrylate can be used. It is also conceivable to use solid substrates covered with a semisolid.

It is conceivable to coat a solid substrate with a semisolid layer. For example, a glass or plastic substrate, in particular a sphere, can be provided with a gel coating, in particular a hydrogel.

The shape of the substrates may, for example, be at least approximately a sphere or cylinder, or be a granulate. Other, particularly production-related forms of the substrate are also possible. For example, surface-enlarged carrier pellets can be used, e.g. made of polypropylene. Carrier pellets usually have a very open shape with webs and a large proportion of perforations in relation to the closed proportion of the carrier pellet. The surface area of the carrier pellet is thereby significantly increased compared to the surface area of the volume into which it can be inscribed with its outer dimensions. For example, the carrier pellets are made of a polyolefin or other plastic, in particular injection molded. Preferably, they have struts in the form of meridians and latitudinal circles of a reduced globe, which enclose an empty interior of it. Preferred is an average ratio of less than ten between the largest and smallest dimension of the substrates. This is advantageous for the removal of the substrates.

The surface, particularly of solid substrates, can be smooth. Preferably, however, the surface is textured. This increases the surface area and also allows for improved retention of organisms on the surface. For example, depressions, which can be punctual or linear, may be in the surface. These can be pores, grooves, furrows, indentations and/or punctual surface depressions, which can be formed in particular with an undercut or a widening in depth. Preferably, the depressions have undercuts. This allows organisms to get a better grip on the surface of the substrates. After harvesting a biofilm, residues may remain in the depressions which can serve as inoculum for inoculating the next production batch.

Preferably at least part, preferably all of the substrates are at least partially translucent, wherein said part of the substrates, preferably all of the substrates are preferably completely translucent and in particular transparent.

In this patent application, transparency is understood to mean the absence of optical opacity of a substrate. In this patent application, partial light transmission means that a substrate is partially opaque and/or just hazy or partially reflective at the inside and/or on the surface such that only part of the light falling on the substrate is transmitted by the same.

An advantage of at least partially transparent substrates is that a bulk material of such substrates refracts incident light for illuminating the organisms multiple times and thus allows a greater depth of penetration of the light into the reactor chamber. Refraction and total reflection can also cause part of the light to change direction significantly. Organisms that are illuminated from the front may additionally be illuminated from behind or the side. This significantly improves the utilization of incident light. The light that passes through a biofilm is not very suitable for illuminating other organisms, as wavelength ranges that the organisms need for growing are filtered out by the biofilm and are no longer available to subsequently illuminated organisms. Therefore, light that comes from a different direction but has not yet passed through a biofilm is particularly valuable for illumination. Such light is distributed particularly well in the bulk material by the transparent substrates.

For example, glass, mineral crystals, plastics or gels can be used as translucent materials for the substrates. A fully transparent substrate material is preferred. Such material makes use of a particularly large proportion of the incident light.

Preferably, the substrates are at least approximately spherical or non-uniform or have at least approximately the shape of a short cylinder or another geometric body with at least approximately similar dimensions in preferably all directions. Preferably, a significant portion of the bulk volume does not fill with the substrate. This can be achieved, for example, with the aforementioned forms of substrates. Through the vacancies in the bulk volume, even in the case of increased growth, light can enter the inner area of the bulk volume or an area further back in the direction of illumination.

Alternatively or additionally, the substrates can be reflective. It is conceivable that the surface of at least part of the substrates is partly reflective and partly transmitting. Reflections on the surface deflect part of the light obliquely. This, as well as the transparency of the substrates, results in more directions from which an illuminating of the organisms takes place as well as a more even distribution of light in the bulk material.

Preferably, translucent substrates have an uneven surface shape. This causes a falling light to be refracted in more directions. This results in a stronger deflection of incident light in directions oblique thereto. A non-uniform surface shape is all the more advantageous the larger the substrates are, particularly if they are on average larger than 5 mm. Substrates that are smaller than 20 mm preferably do not have an uneven surface shape. An uneven surface shape can be achieved, for example, by a surface treatment of the substrates. For example, a forming, scoring, embossing, chipping or roughening or primary shaping can be carried out, creating the irregular surface shape.

The substrates are preferably biodegradable. This has the advantage that the substrates can be applied as fertilizer in agriculture after removal from the bioreactor. Suitable materials for this are in particular organic polymers, e.g., cellulose, carrageen, nanocellulose, alginate, or polymers with biodegradable predetermined breaking points and/or compounds.

The bioreactor preferably has a drying device, the activity of which allows the biofilm to be dried out. For example, the drying device may be part of the humidifying device. The humidifying device may be controlled by the drying device in such a way that drying is achieved through the absence of moisture. A drying phase alternating with a growth phase, in contrast to a continuous method, in which one end of a reactor is watered and another end is dried, allows for greater efficiency in biomass production, as the period of the drying process means less loss of productivity than the continuous non-use of a part of the reactor for growth.

Preferably, the bioreactor comprises an extraction device, the activity of which allows the at least one part of the biofilm in the bioreactor to be extracted from the bioreactor with and/or without substrates. For example, the extraction can be carried out remotely by activating the extraction device. The extraction device can have an opening device that can open the bioreactor. The extraction device can have a mechanical moving device, in particular a shaking device and/or a stirring device and/or a device for introducing compressed air, by which the bulk material in the bioreactor may be for shaken, stirred, blown through or otherwise moved. In this way, biofilm may be detached from the substrates. The bioreactor preferably comprises a substrate filter which keeps at least the substrates inside the reactor chamber while organisms and/or organic material such as biofilm and/or substances produced by the organisms can leave the reactor chamber through the extraction device.

Preferably, the bioreactor has a removal device. This is preferably located at a lower end of the bioreactor. Biofilm with and/or without substrates can be removed by the removal device. Preferably, the biofilm is taken out of the tank by gravity, possibly assisted by shaking, and/or by a flushing medium, in particular a gas, preferably air. The removal device is preferably set up to be able to open and close the bioreactor.

In another aspect of the invention, tablets of substrates are proposed. The tablets may be enriched with nutrients. The substrates are preferably made of a gel, which is present in the tablet in dried form. In principle, the types of gels mentioned in this patent application may be used for this purpose. Substrates made of polymers such as Na-polyacrylate are particularly suitable. Such tablets can be transported inexpensively and moistened on site for use in the bioreactor. Preferably, such a tablet is equipped with the species of the organism(s) with which biomass is to be produced. Preferably, the bioreactor is equipped with the tablets and the tablets are humidified inside the bioreactor. Production can then begin.

A light device of the bioreactor may be, for example, an active lighting or a transparent wall of the reactor chamber through which light can enter the reactor chamber from the outside. The sun can also serve as a light device. Preferably, white light is used, but it is also conceivable to use green and/or red and/or blue light. Particularly preferred is daylight, for example, sunlight or mitigated sunlight.

Preferably, the bioreactor has at least one chamber gate. In particular, this is formed and intended so that substrates and/or the organisms may be introduced into the reactor chamber and/or removed from the reactor chamber through the chamber gate. Preferably, the chamber gate is located in an upper portion of the bioreactor. In this way, the bioreactor can be easily filled with substrates through the chamber gate.

Preferably, the substrate filter is a sieve. The mesh size of the sieve is preferably smaller than a large part of the substrates. The chamber gate preferably enables at least approximately contamination-free loading of the reactor chamber with substrates and/or removal from the reactor chamber. The reaction chamber can have several chamber gates. A chamber gate can be designed as a sluice with two successively arranged openable and closable accesses to the reaction chamber.

The gassing device is configured to supply the bioreactor with gas. This can be, for example, air or an exhaust gas or a mixture of air with other gases, such as carbon dioxide or exhaust gas containing carbon dioxide. An exhaust gas can originate in particular from a combustion process or a procedural process.

In order to achieve a certain metabolism of organisms, conditions that represent stress factors for the organisms can be created in the bioreactor. This may take place, for example, by introducing dry and/or dehumidified air, oxygen, nitrogen or toxic gases.

It is also conceivable to add gases that serve to fertilize the microorganisms, such as sulfur oxide and/or sulfur dioxide and/or nitrogen oxide. For fertilization, particulate matter may be present in the gas. The particulate matter can be extracted from the environment and/or present in an air flow and/or exhaust gas flow and/or selectively added. These can be part of an exhaust gas.

Preferably, the gassing device comprises a gas inlet leading into the reactor chamber and a gas outlet leading out of the reactor chamber. Preferably, the gassing device comprises a gas conveying device. A gas conveying device may be located before the reactor chamber or after the reactor chamber with respect to the gas flow being conveyed through the reactor chamber This can be a fan or compressor. Its pressure side can be connected to the gas inlet. Alternatively or additionally, in the case of a second compressor, this can be connected to the gas outlet on the suction side. With suitable throttling at the gas inlet, negative pressure can then be set in the reactor chamber. Alternatively, positive pressure can be set with a suitable throttling at the gas outlet if a fan or compressor is connected to the gas inlet. By adjusting the pressure conditions in the reactor chamber, the growth of the organisms can be influenced.

The humidifying device supplies the reactor chamber with moisture. The humidifying device can cause drops of a humidifying liquid to flow through the bulk material in the direction of gravity. In particular, the humidifying device may have a drop outlet. Alternatively or additionally, the humidifying device may generate aerosol which can be introduced into the gas of the gassing device. Then, the aerosol can be transported through the reactor chamber with the gas flow that is conveyed by the gassing device. Preferably, the droplets of the aerosol are significantly smaller than an average diameter of a through-path, for example less than one tenth of it. The average diameter of the through-path can be assumed to be one fifth of the average substrate size. Preferably, the average droplet size is less than 1/50 of the average substrate size. A preferred average droplet size is thus less than 200 pm for an average substrate size of 10 mm, for example. Average droplet sizes of 20 to 100 pm are preferably used. Droplet generation can be done conventionally by a droplet generator such as a nebulizer. The droplet generator can use available methods according to prior art, such as ultrasound, valves or fine meshes. The droplets can be generated in such a way that they enter the gas flow of the gassing device after being generated. The gas flow from the gassing device is preferably split into a plurality of small partial gas flows for each of the plurality of through-paths after entry into the reactor chamber. Preferably, each of the partial gas streams carries aerosol.

Through precipitation of droplets or condensation along a through-path, the organisms are supplied with moisture. Excess liquid can flow along the through-channels in the direction of gravity. Preferably, the gas inlet is further in the direction of gravity than the gas outlet. As such, the flow of liquid can flow by gravity at least partially against the locomotion of the partial gas flows. Preferably, the liquid that exits the bulk material in the direction of gravity reaches the droplet generator again. Preferably, droplets that exit from the bulk material in the direction of the gas flow are stored together in a droplet extractor to form larger droplets and/or a liquid flow. Preferably, at least approximately droplet-free gas emerges from the bioreactor. These drops and/or the liquid flow preferably re-enter the bulk material in the direction of gravity and moisten it by flowing through in the direction of gravity. These drops and/or the liquid flow can reach the droplet generator again. The droplet generator can add the liquid to the gas flow again. Preferably, approximately no liquid leaves the bioreactor. It is conceivable to use an air dryer in the gas flow before it leaves the bioreactor to extract air humidity and return it to the bioreactor.

Preferably, the humidity in the bioreactor is controllable by means of the humidifying device by means of a control or regulating device or adjustable through the feedback of a humidity measurement information. In particular, a droplet generator can be controlled to increase or decrease the release of liquid in the form of droplets. Preferably, the biomass production process can be guided by controlling or regulating the humidity in the bioreactor. For example, high humidity can be set during a growth phase, and low humidity can be set during a drying phase before harvesting. Preferably, the humidifying device cooperates with the gassing device to control or regulate the humidity. The gassing device can therefore also be controlled or regulated by the control or regulating device.

The liquid is preferably water. However, it is conceivable to supply organisms, that use a liquid other than water in their metabolism, with this liquid. The liquid can be collected in the surroundings, for example as rainwater or condensation water, and stored for humidification of the bioreactor. Alternatively or additionally, it can be supplied from a liquid supply that is carried out from a central reservoir through pipelines, such as from a water supply network. Nutrients for the organisms may be added to the liquid. Nutrients may be, for example, glucose, molasses, basic building blocks for metabolism, alcohol, acetone. In addition, nutrients can be taken from the substrate, particularly inorganic nutrients.

In particular, a pH buffer can be added to the liquid, particularly water. It is also possible that the pH buffer be present in the substrate. It is also conceivable to add a pH buffer to the supplied gas. A pH buffer can be, for example, a carbonate buffer, an acetate buffer, a phosphate buffer or any other known buffer.

One way to achieve a specific metabolism of organisms is to set growth-promoting humidity conditions or dryness in the bioreactor. Preferably, the bioreactor is configured for this purpose. Preferably, the bioreactor is set to a humidity of 70 to 100% to promote the growth of the organisms.

The light device may have active artificial lighting for the organisms. Alternatively or additionally, at least a section of a wall of the reactor chamber, preferably a large part of the reactor chamber or the entire reactor chamber, can be formed translucent. In this way, ambient light, particularly sunlight, can enter the reactor chamber and illuminate the organisms.

Preferably, the reactor chamber is tubular. Particularly preferably, the reactor chamber comprises a transparent pipe. The longitudinal axis of the pipe in operation is preferably oriented obliquely to and particularly preferably at least approximately in the direction of gravity. The reactor chamber can alternatively be part of a flatbed reactor. The flatbed is preferably oriented vertically. The bulk material has, in the main illumination direction, a dimension of preferably 6 to 40 cm, more preferably less than 20 cm and particularly preferably less than 10 cm, particularly, but not exclusively, in the case of a flatbed reactor. It is conceivable to form the reactor chamber in such a way that the organisms can be illuminated from several different directions from the surroundings. It is also conceivable that the reactor chamber be designed to be rotated during operation. In particular, a tubular reactor chamber can be rotatable about its longitudinal axis, such as in the manner of a rotatable advertising pillar. In this way, the dimension of the bulk material in the main illumination direction can be twice the penetration depth of the light. This allows for a more compact and comparatively less elongated or flat structure of the bioreactor.

Preferably, the temperature in the bioreactor is settable. Preferably, the setting range is between 15 and 50° C. Alternatively, the bioreactor can operate in a smaller temperature range or at a certain constant temperature, which can, in particular, be adapted to an available heat source. Preferably, waste heat is used to heat the bioreactor. Particularly in cold outdoor temperatures, the production capacity of the bioreactor can be increased as a result. The waste heat can be, for example, room heat escaping from a building, particularly if the bioreactor is used on a façade of the building. It is also possible to use waste heat from technical processes, such as from machines or heated room air, waste heat from air-conditioning systems, waste heat from combined heat and power plants or waste heat from charging stations for electric cars. Also, waste heat with low temperatures up to about 50° C. from large-scale technology such as power plants, cement works, steelworks, aluminum works, asphalt works and the like can generally be used, as can waste heat from animal husbandry. Waste heat with temperatures below 50° C., particularly below 40° C. or below 30° C., but in any case above the ambient temperature, can be used sensibly in this way. Since it is difficult to use elsewhere, it is available in many places. The temperature in the bioreactor can be adjustable. Conventional methods can be used for this purpose.

The above features of bioreactors can be used in combination, provided this is not technically impossible.

In another aspect of the invention, a lantern is proposed, comprising a bioreactor according to one of the described embodiments. A light device with active lighting of the bioreactor can serve both as illumination for the organisms at night and illuminate objects in the vicinity of the lantern.

In another aspect of the invention, a window for a building is proposed, comprising a bioreactor according to any of the described embodiments. The bioreactor can be used in an portion of the window that is exposed to the temperature of the room on the inner side of the window. In this way, the bioreactor may use daylight incident from the outside of the building. At the same time, it can be exposed to a favorable operating temperature in many cases. At night, additional artificial light from the room on the inner side of the windows can be used to illuminate the bioreactor.

In another aspect of the invention, a charging station for electric vehicles is proposed, comprising a bioreactor according to one of the described embodiments. Waste heat from the charging station can be fed into the bioreactor in order to heat it.

In another aspect of the invention, a method of producing biofilm with a bioreactor according to one of the described embodiments is proposed.

In one embodiment of the method, the organisms are fed and/or fertilized with fine particulate matter.

In a further embodiment of the method, the bioreactor is heated with waste heat.

In a further embodiment of the method, the bioreactor is controlled in such a way that the organisms that form a biofilm on the substrates dry out after a growth phase inside the reactor chamber. This can be achieved, for example, by supplying non-humidified gas. It is also possible to increase the temperature in the reactor chamber. One advantage of this method is that the generated biofilm does not have to be removed from the reactor chamber together with the substrates.

In another embodiment of the method, generated, dried biofilm is removed from the reactor chamber while substrates remain in the reactor chamber. Preferably, the dried biofilm is detached from the substrates before removal from the reactor chamber, for example by moving, in particular shaking and/or stirring, and/or spinning and/or turning over the reactor chamber so that the top side is turned downwards, in particular several times, and/or by whirling due to strong gas input, in particular pulsating gas input. When using substrates comprising gel, the detaching of the biofilm from the substrates is facilitated by the fact that the gel contracts during drying and detaches at least partially from the surface of the biofilm. The biofilm can be extracted from the bioreactor e.g., by gravity, possibly by shaking, pressure surge, blowing out or supported by other movement of the contents of the reactor chamber. The removal can be done through a sieve that retains the substrate, or through blowing out. In particular, the gassing device can be used for blowing out. For this purpose, this can be run in a different operating mode. Preferably, in this case, the dried biofilm is withdrawn from the gas stream exiting the reactor chamber. An extractor according to prior art can be used for this purpose.

In a further embodiment of the method, the substrates are removed from the reactor chamber together with the biofilm generated by them. The contents of the bioreactor may or may not have been previously dried. The biofilm may at least partially adhere to the substrates, particularly if they are not dried. Not drying the contents has the advantage that the bioreactor can be used again, immediately after the substrates have been removed, for the growth process of organisms on other substrates. The generated biofilm can then be removed from the substrates outside the reactor chamber. In particular, the removed substrates can be dried outside the reactor chamber for this purpose. After removing the biofilm from the substrates, they can be used again in the reactor chamber. Preferably, the substrates have structures in which a residue of the biofilm remains, which can serve as an inoculum when used again. In particular, such structures can be undercuts or depressions. In this way, the reactor does not need to be inoculated again.

In a further embodiment of the method, dried substrates are introduced into the reactor chamber. These can be in tablet form. Preferably the tablets are inoculated. This can be done when the tablets contain a residue of a biofilm previously grown on the substrates as inoculum. Substrates can be moistened within the biofilm. This allows them to reach the size they have when moist. After humidifying, the bioreactor can be used for production.

In another aspect of the invention, a method for fertilizing in agriculture is proposed. The method comprises the production of biofilm on biodegradable substrates with a bioreactor according to the invention. In a further step of the method, the substrates with the biofilm are removed from the bioreactor and spread for fertilization in agriculture, particularly in the vicinity of plants to be fertilized. This can also be seen as using produced organisms and/or biofilm. In particular, nitrogenous organisms grow in the bioreactor. In particular, these organisms are able to separate nitrogen from a gas, particularly atmospheric nitrogen. Such organisms can be, for example, cyanobacteria, in particular cyanobacteria having heterocysts, or other nitrogen-fixing bacteria, for example rhizobial species and/or actinomycetes of the genus Frankia.

In a development of the method described in the last paragraph, this can be extended to use substrates that can absorb water, whereby the substrates with the produced organisms are spread on or in soil for plant growth so that the soil is improved. It is then, in addition to its property of being supplied with fertilizing organisms, able to store water in an improved way.

In a further aspect, it is proposed to use biofilm and/or organisms generated by one of the methods described above and/or with a bioreactor described above as food for humans and/or animals. For this purpose, substrates that are edible for humans and/or animals can be used in the method or the bioreactor. The substrates with the organisms and/or biofilm then result in edible portions. This can also be regarded as the production process of such portions.

The drawings in the Appendix show exemplary embodiments of the invention in which:

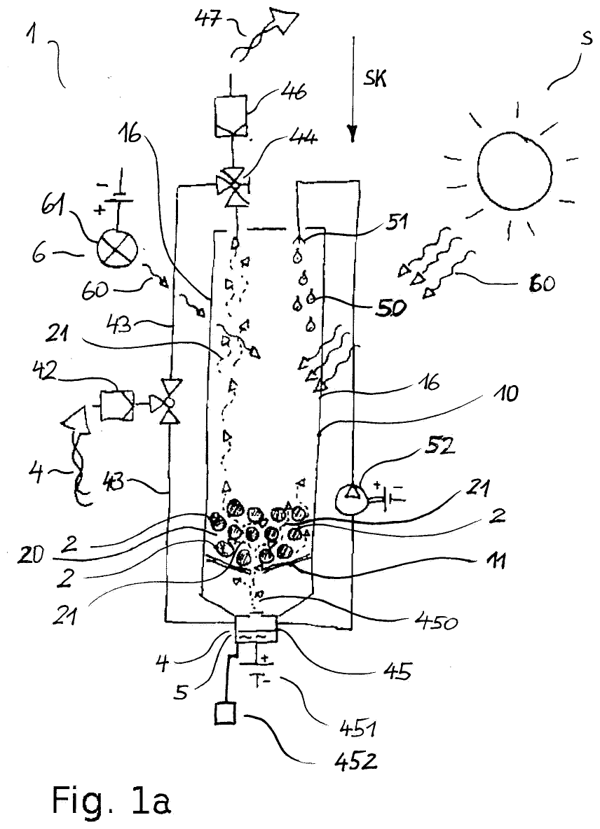

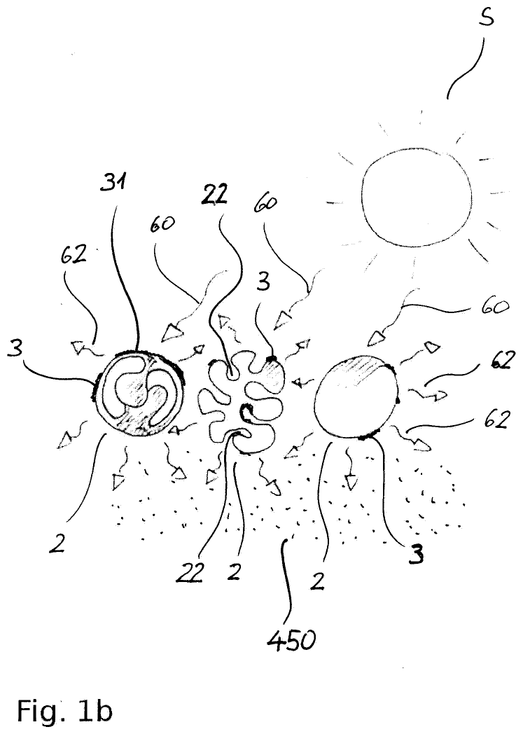

FIG. 1a schematically shows a sketch of a bioreactor according to the invention,

FIG. 1b schematically shows a sketch of part of a bulk material of substrates in the bioreactor,

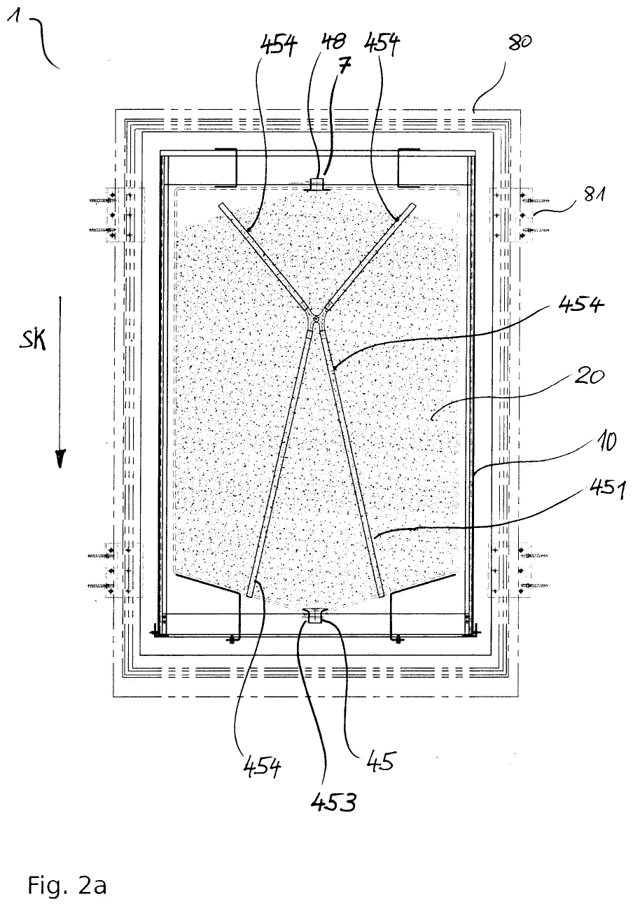

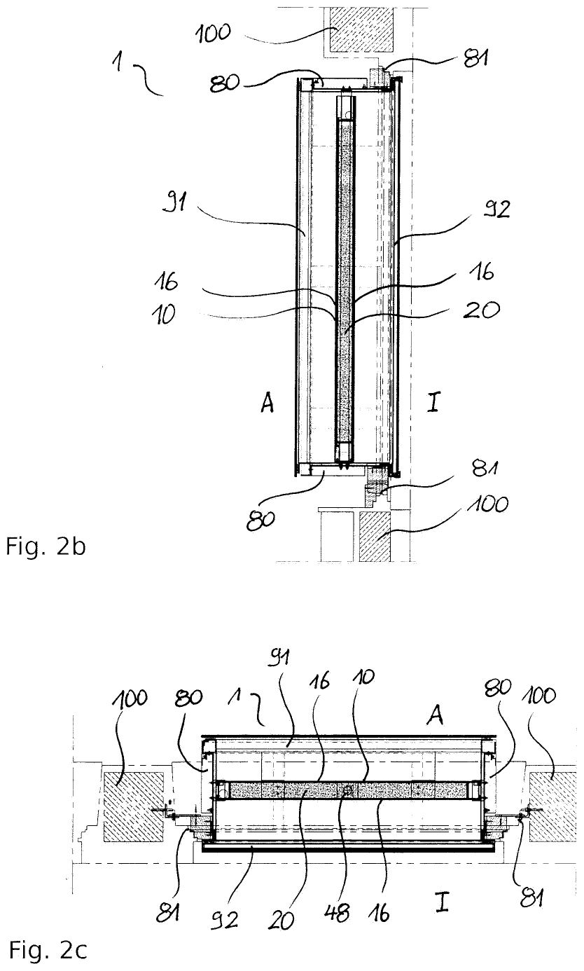

FIG. 2a is a drawing of a bioreactor in frontal view, which is realized as a facade element,

FIG. 2b shows the bioreactor from FIG. 2a in a side view, and

FIG. 2c shows the bioreactor from FIG. 2a in a top view.

FIG. 1 shows a schematic side view of a bioreactor 1 according to the invention. The Bioreactor 1 comprises a reactor chamber 10. Substrates 2 are arranged in the reactor chamber. The substrates 2 form a bulk material 20 within the reactor chamber 10. The bulk material 20 rests on a support 11 inside the reactor chamber 10. The bulk material 20 is shown schematically and may in practice extend to the top end of the interior of the reactor chamber 10. Below the support 11, an aerosol device 45 is arranged, which forms a combination of a gassing device 4 and a humidifying device 5. The aerosol device 45 may be supplied with electrical energy from a power supply 451, which may be a battery. The aerosol device 45 comprises a nebulizer for liquid, in particular water, which is not explicitly shown, and an opening leading interior the reactor chamber 10. Aerosol 450 rises from the aerosol device 45 into the interior of the reactor chamber 10. This enters through the support 11 into the bulk material 20. There are through-paths 21 between the individual substrates 2. The aerosol 450 can pass through the through-paths 21. Through-paths 21 are also shown schematically in the upper part of the reactor chamber 10, in which the bulk material 20 can also be located, but this is not shown in FIG. 1. As it passes through, the aerosol 450 releases liquid to the substrates 2 or to organisms 3 that grow on the substrates 2 and are not explicitly shown in FIG. 1.

The aerosol device 45 may optionally be connected to a drying device 451. The drying device 451 may cause the aerosol device 45 to no longer discharge aerosol 450.

Alternatively or additionally, aerosol 450 may optionally be actively separated from an inflow or outflow to or from the bioreactor 1, which is not explicitly shown in the figures. For this purpose, e.g. a cold trap or an absorber can be used. It is also possible to passively separate aerosol, for example by enlarging the surfaces alongside of which the inflow or exhaust flow of aerosol is guided. If the organisms are to be dried, the drying device 451 can be used to separate aerosol 450 from the inflow. In normal growth mode, in which aerosol 450 is usually added to the inflow, aerosol discharged from the exhaust flow can be separated and recycled. In this way, the bioreactor consumes less water. For example, the drying device 451 can be used for separation and alternatively, a separately provided device can be used for this purpose.

A partially closed gas loop may be provided particularly preferably in embodiments of the bioreactor by supplying at least a part of the exhaust flow to the inflow, which is not explicitly shown in the figures. Usually, fresh gas is constantly flushed in to supply the organisms. The removed exhaust gas can be freed of escaping aerosol 450 by means of a drying device 451. The water obtained in this way can be recycled, which reduces the water consumption of bioreactor 1.

In this way, drying of the organisms 3 present in the reactor chamber 10 or of biofilm 31 containing them can be achieved. Gas can then continue to be passed through the bioreactor 1, in particular dry gas such as dry air. Due to the permanent humidifying of the substrates 2 or organisms 3 located on them, droplets from the aerosol device 45 can accumulate to form larger droplets or a trickle, wherein the water follows gravity SK and runs over the bulk material 20 to the support 11, from where the liquid is returned to the aerosol device 45 and is nebulized again. The aerosol device 45 may be connected to an external supply of liquid, but is not shown in FIG. 1.

In the embodiment shown, a fluid pump 52 has its suction side connected to the aerosol device 450. The liquid pump 50 delivers water from the aerosol device 450 to a drip device 51. The drip device 51 can release drops 50. The drops 50 drop into the bulk material 20. This liquid, which runs through the bulk material 20 due to gravity SK, moistens the bulk material 20. In particular, the upper part of the reactor chamber 10 can be moistened in this way. The liquid pump 52 and the drip device 51 form part of the humidifying device 5. The part of the humidifying device 5 described in this paragraph can also be used in other embodiments.

In the embodiment shown, the gassing device 4 comprises a gas supply device 42 through which gas 4 can be supplied to the bioreactor, in particular ambient air. The gas supply device 42 comprises a filter and/or a sterilizer for gas entering the bioreactor, which largely prevents contamination of the bioreactor 1. The gas 4 provided by the gas supply device can enter a gas return line 43. In the gas return line 43, gas is returned to the aerosol device 45, which has previously passed through the bulk material 20 in the reactor chamber 10 as an aerosol. A gas conveying device is not explicitly shown. It is possible for the aerosol device 45 to take over the gas conveying. In particular, it comprises a blower that conveys gas to the reactor chamber. The gas flow through the reactor chamber 10 reaches a gas distributor 44 after exiting the reactor chamber 10. The gas distributor 44 directs part of the gas arriving there into the gas return line 43 and another part to a gas discharge device 46. The gas discharge device 46 may comprise a filter and/or a sterilizer. By discharging exhaust gas 47 from the bioreactor, preferably no or only little contamination takes place. Through a constant supply of fresh gas 4, it is possible for the bioreactor 1 to be supplied with gases that are necessary for the growth of the organisms 3.

A wall 16 of the reactor chamber 10 is preferably made of transparent material. Then, light 60 from the sun S can penetrate through the wall 16 into the interior of the reactor chamber 10. Alternatively or additionally, artificial lighting 61 may also emit light 60 which may penetrate through a transparent wall 16 of the reactor chamber 10 into its interior. The artificial lighting 61 may be connected to a power supply. This can be a battery, among other things. Preferably, the wall 16 of reactor chamber 10 is designed to be tubular. Preferably, the entire wall 16 is transparent.

FIG. 1b schematically shows a few different substrates 2 that are illuminated by the sun S. The substrates 2 are translucent.

On the far right of FIG. 1b, a substrate 2 with a smooth surface is shown. The substrate 2 is hit by light 60 from the sun S. Due to refraction, total internal reflection and scattering by irregularities of the surface, the incident light 60 is emitted from substrate 2 as scattered light 63 in a multitude of directions. The scattered light can in turn fall onto other substrates 2 so that organisms 3 on them can be illuminated with it.

The image of the middle substrate 2 shows a highly textured substrate 2. This substrate 2 has a considerably increased surface area compared to a sphere of the same external dimension. It shows strong indentations. In particular, this substrate 2 comprises undercuts 22 in its interior which are open to the exterior of the substrate 2.

The illustration of the left substrate 3 schematically shows a substrate 2 that has a twisted structure by twisting of an initial body of a twisted structure. This increases the surface area of substrate 2 compared to the surface area of the initial body.

The substrates 2 show beginning growth of organisms 3. In particular, organisms 3 may be present in the undercuts 22. Even after the biofilm has been harvested from the organisms 3, organisms remain in the undercuts 22. These can serve as inoculum when the substrates 2 are used again in a bioreactor 1 according to the invention.

FIG. 2a shows a front view of bioreactor 1, which is constructed as a flatbed reactor. The bioreactor 1 is designed as a facade element. Bioreactor 1 comprises a reactor chamber 10 within which a bulk material of 20 substrates 2 is arranged. The reactor chamber 10 comprises an aerosol device 45. The aerosol device 45 is arranged below the bulk material 20 as seen in the direction of gravity SK. Inside the reactor chamber 10, an aerosol distribution device is arranged which has several, in particular as shown four, aerosol outlet pipes 454. The aerosol outlet pipes 454 have openings around their circumference from which aerosol 450 can escape. The aerosol outlet pipes 454 are supplied with aerosol by the aerosol device 45. The aerosol distribution device 451 has the purpose of distributing aerosol 450 over a distance not covered by the bulk material 20. In this way, it can be prevented that aerosol droplets are no longer present in sections of the though-paths 21 that are far away from the aerosol device 45. The length of the through-paths 21 in the bulk material 20 may be shortened compared to an arrangement without aerosol distribution device 451. The reactor chamber 10 preferably comprises a gas outlet 48 arranged at an end of the reactor chamber opposite the aerosol device 45. From there, gas can be returned to the aerosol device 45. Through the opening through which the aerosol device 45 introduces aerosol 450 into the reactor chamber 10, substrates 2 of the bulk material 20 can be withdrawn from the reactor chamber 10. In such a variant, the aerosol device 45 can also form an extraction device 453. The reactor chamber 10 can be filled with substrates 2 through the gas outlet 48. In such a variant, the gas outlet 48 can also form a chamber gate 7 at the same time.

The reactor chamber 10 is preferably attached to a frame 80. The frame 80 preferably comprises fastening elements 81 for fastening the frame 80 to a facade or another building part.

The bioreactor 1 preferably generally follows the function scheme shown in FIG. 1a.

Features of this embodiment may also be used in other embodiments, in particular the aerosol distribution device 451.

FIG. 2b shows the bioreactor 1 from FIG. 2a in a section of a side view in an installation situation in a building wall 100. The reactor chamber 10 with the bulk material 20 is arranged between two glass panes 91 and 92. The reactor chamber 10 has transparent walls 16 between which the bulk material 20 is arranged. Through the walls 16, artificial light from the inside of the building can fall from the inner side of the building and daylight from the outside of the building can fall from the outer side onto the bulk material 20. The glass panes 91 and 92 are held in the frame 80. The frame 80 is connected to building wall 100 via connecting elements 81. The building wall 100 comprises a cut-out in which the bioreactor 1 is arranged.

FIG. 2c shows a top view of the bioreactor from FIGS. 2a and 2b. Same features are designated with the same reference signs. FIG. 2c also shows the gas outlet 48. Through this gas outlet 48, the reactor chamber 10 can be filled with substrates 2.

1-22. (canceled)

23. A bioreactor with several substrates formed of a solid or a semi-solid material for immersed growth with aerophilic, photosynthesizing organisms for use with a device providing light for the organisms, wherein the bioreactor comprises
a reactor chamber in the interior of which the substrates are arranged for growth with the organisms,
a gassing device for supplying the organisms with gas, and
optionally wherein the reactor responds to the organization of organisms with moisture, wherein the substrate can be replaced using the switch in the reactor chamber, and the substrates can be used for gas and/or fire, and the bioreactor is set up so that the bed can be illuminated with light from the lighting device.

24. The bioreactor of claim 23, wherein the substrates have an average size of more than 1 mm.

25. The bioreactor of claim 23 comprising hydrogel.

26. The bioreactor of claim 23, wherein the substrate is structured.

27. The bioreactor of claim 23, wherein the substrate is translucent or transparent.

28. The bioreactor of claim 23, wherein the substrate is degradable.

29. The bioreactor of claim 23 further comprising a drying device for the organisms or the entire biofilm in the bioreactor.

30. The bioreactor of claim 23 further comprising a stripping device to remove at least some of the organisms in the bioreactor with and/or without substrates which can be removed from the bioreactor.

31. The bioreactor of claim 23 wherein the bioreactor has at least one chamber port for introduction or removal of substrates and/or organisms into the reactor chamber.

32. A substrate for the immersion bioreactor of claim 1 wherein, after it is used, the substrate is placed on an outer surface comprising hydrogel.

33. The biofilm or organism on the substrate of claim 32, dried and formed into at least part of a tablet.

34. A method wherein organisms and/or biofilm are produced with the bioreactor of claim 1.

35. The method according to claim 34, wherein the bioreactor is controlled so that the organisms form a biofilm on the substrates and dry out after a growth phase within the reactor chamber.

36. The method according to claim 34, wherein the dried biofilm is removed from the reactor chamber while substrates remain in the reactor chamber.

37. The method of claim 36, wherein the dried organisms are detached from the substrates before being removed from the actuator chamber.

38. The method according to claim 36, wherein the dried biofilm is detached from the substrates before being removed from the actuator chamber.

39. The method of claim 36, wherein the substrates are removed from the actuator chamber, together with the organisms produced on them.

40. The method of claim 34 wherein the dried substrates are introduced into the reactor chamber for the production of organisms or biofilm.

41. A method of fertilizing or increasing water absorption comprising applying the biofilm or organisms of claim 33 on or into a medium for plant growth.

42. The biofilm and/or organisms of claim 34 wherein the organisms are grown on edible substrates for use as food.
